(19) United States
(12) Patent Application Publication
(10) Pub. No.: US 2007/0243249 A1
(43) Pub. Date: Oct. 18, 2007

(54) Novel formulation of pyridoxal-5'-phosphate and method of preparation

(76) Inventors: Albert D. Friesen, Winnipeg (CA); John Carter, Keswick (CA)

Correspondence Address: MERCHANT & GOULD PC, P.O. BOX 2903, MINNEAPOLIS, MN, 55402-0903, US

(21) Appl. No.: 11/288,971

(22) Filed: Nov. 28, 2005

Related U.S. Application Data
(60) Provisional application No. 60/630,574, filed on Nov. 26, 2004.
(60) Provisional application No. 60/690,127, filed on Jun. 14, 2005.

(51) Int. Cl. A61K 9/20; A61K 31/675; A61K 9/28

(52) U.S. Cl. 424/465; 424/474; 514/350

(57) ABSTRACT

A pyridoxal-5′-phosphate pharmaceutical formulation suitable for oral administration is provided comprising a dissolution profile, when measured in a standard dissolution apparatus, according to the United States Pharmacopoeia dissolution test, at 37° C. in a 0.05M phosphate buffered solution having a pH of 6.8 at 75 rpm, as follows: (a) greater than about 30% at 15 minutes, (b) greater than about 85% at 30 minutes, (c), greater than about 90% at 45 minutes, or (d) greater than about 95% at 60 minutes. Additionally, in vivo oral intake of between 15 and 60 mg/kg of a pyridoxal-5′-phosphate pharmaceutical formulation can produce a maximum plasma level (Cₘₐₓ) of between about 1 mg/L and 8 mg/L. A pharmaceutical formulation provided comprises (a) a core, wherein said core comprises pyridoxal-5′-phosphate or a pharmaceutically acceptable salt thereof; (b) a sub-coat surrounding the core; and (c) an enteric coat surrounding the sub-coat.

This application claims the benefit under 35 U.S.C. §119(e) of U.S. Provisional Application No. 60/690,127, filed Jun. 14, 2005, and U.S. Provisional Application No. 60/630,574, filed Nov. 26, 2004, the entire disclosures of which are hereby incorporated by reference.

### FIELD OF INVENTION

The present invention relates to pharmaceutical formulations of pyridoxal-5′-phosphate and methods of preparing the same.

### BACKGROUND

Pyridoxal 5′-phosphate is useful for the treatment and prevention of a variety of diseases such as hypertension, cerebrovascular disorders, cardiovascular disorders and diabetes. See for example U.S. Pat. Nos. 6,051,587; 6,417,204; 6,548,519; 6,586,414; 6,605,612; 6,667,315; 6,780,997; 6,677,356; 6,489,348; and 6,043,259. Pyridoxal 5′-phosphate is commercially available in a variety of doses. However, currently available supplements generally deliver lower doses of pyridoxal 5′-phosphate which are too low for the treatment of hypertension, cerebrovascular disorders, cardiovascular disorders and diabetes. As such, it is often necessary for the supplement to be administered several times daily in ordered to achieve suitable therapeutic levels.

### SUMMARY OF INVENTION

The present invention provides novel oral pharmaceutical compositions capable of delivering increased amounts of pyridoxal 5′-phosphate as compared to prior art formulations. The present invention also provides novel pharmaceutical compositions which overcome gastrointestinal side effects associated with the intake of high doses of pyridoxal 5′-phosphate.

In an embodiment, a pyridoxal-5′-phosphate pharmaceutical formulation suitable for oral administration comprises a dissolution profile, when measured in a standard dissolution apparatus, according to the United States Pharmacopoeia dissolution test, at 37° C. in a 0.05M phosphate buffered solution having a pH of 6.8 at 75 rpm, as follows: (a) greater than about 30% at 15 minutes, (b) greater than about 85% at 30 minutes, (c), greater than about 90% at 45 minutes, or (d) greater than about 95% at 60 minutes. Additionally, in vivo oral intake of between 15 and 60 mg/kg of an embodiment of the pyridoxal-5′-phosphate pharmaceutical formulation can produce a maximum plasma level (Cₘₐₓ) of between about 1 mg/L and 8 mg/L.

In an embodiment, the pharmaceutical formulation comprises (a) a core, wherein said core comprises pyridoxal-5′-phosphate or a pharmaceutically acceptable salt thereof; (b) a sub-coat surrounding the core; and (c) an enteric coat surrounding the sub-coat. Embodiments may be incorporated into any suitable oral dosage form, such as a tablet or a capsule.

In an embodiment, the formulation comprises at least 50% w/w pyridoxal-5′-phosphate, or a pharmaceutically acceptable salt thereof.

An embodiment includes a method of producing an embodiment of a pyridoxal-5′-phosphate pharmaceutical formulation comprising a pre-blend of at least 50% w/w pyridoxal-5′-phosphate.

An embodiment includes a method of administering an embodiment of the pyridoxal-5′-phosphate pharmaceutical formulation can promote patient compliance.

### BRIEF DESCRIPTION OF THE FIGURES

FIG. 1 is a flow chart illustrating the steps in preparing a granulating preparation for use in the production of enteric coated tablets.

FIG. 2 is a flow chart illustrating the steps in preparing a semi-final blend preparation and a final tableting preparation and tablet cores for use in the production of enteric coated tablets.

FIG. 3 is a flow chart illustrating the steps in coating tablet cores to provide enteric coated tablets.

FIG. 4 is a line graph illustrating the dissolution profile enteric coated tablets versus dissolution of the core of the tablet.

FIG. 5 is a line graph illustrating the dissolution profile enteric coated tablets versus dissolution of the core of the tablet.

FIG. 6 is a line graph illustrating the dissolution profile enteric coated tablets versus dissolution of the core of the tablet.

FIG. 7 is a line graph illustrating the dissolution profile enteric coated tablets versus dissolution of the core of the tablet.

FIG. 8 is a graph illustrating the low, high, and average % release values for enteric coated tablets.

### DETAILED DESCRIPTION

### DEFINITIONS

The term “percentage weight per weight (% w/w)” refers to the weight percentage of the particular compound or excipient relative to the total weight of the composition of which the compound or excipient is a constituent of.

The term “percentage weight per volume (% w/v)” refers to the weight percentage of the particular compound or excipient relative to the total volume of the solution of which the compound or excipient is a constituent of.

The term “particulate” refers to a state of matter that is characterized by the presence of discrete particles, pellets, beads, or granules irrespective of their size, shape, or morphology.

The term “multiparticulate” as used herein means a plurality of discrete, or aggregated, particles, pellets, beads, granules, or mixtures thereof irrespective of their shape, size, or morphology.

The term “disintegrant” as used herein means any substance used in solid dosage forms to promote the disruption of the solid mass into smaller particles which are more readily dispersed or dissolved.

The terms “binding agent” or “binder” as used herein means any substance that helps hold a tablet together. A binding agent” or “binder” includes any substance used to cause adhesion of powder particles in tablet granulations.

The term “lubricant” as used herein means any substance used in tablet formulations to reduce friction during tablet compression. The term “lubricant” also includes any substance which permits the compressed tablet to be properly ejected from a tableting machine.

The term “glidant” as used herein means any substance used in tablet formulations to reduce friction during tablet compression or any substance which are used to facilitate the flow of the powders in the tableting process.

The term “anti-adherent” as used herein means any substance which prevents the sticking of tablet formulation ingredients to punches and dies in a tableting machine during production.

The term “exicipient” as used herein means any inert substance combined with an active drug in order to produce a drug dosage form.

The term “colorant” as used herein means any substance used to impart color to pharmaceutical preparations (e.g., tablets).

The terms “sub-coat”, “seal coat” or “sealing coat” as used herein refers to any protective coating and include coatings which are moisture or solvent resistant.

The terms “enteric coat” or “enteric coating” as used herein, means any coating or shell placed on a tablet that breaks up and releases the drug or active ingredient into the intestine rather than the stomach.

Use of a pharmaceutical composition according to the invention facilitates patient compliance. There is an inverse relationship between patient compliance and frequency of the intake of the medication. The higher the frequency of intake of a prescribed medication, the lower the rate of compliance. Patient compliance decreases when the prescribed medication is difficult to administer and consumption of the medication is associated with physical discomfort. Pharmaceutical compositions according to the invention promote patient compliance as compositions provide high doses of pyridoxal 5′-phosphate in a single or twice daily oral dosage form which is sized for easy swallowing.

A limiting factor in the tolerance to high doses of pyridoxal 5′-phosphate is gastrointestinal discomfort characterized mainly by nausea and vomiting. Embodiments of the present invention provides novel pharmaceutical compositions suitable for the oral administration of high doses of pyridoxal 5′-phosphate with minimal gastrointestinal side effects. Furthermore, controlled release assists in maintaining a therapeutic concentration of drug in the body for an extended period of time by controlling its rate of delivery.

An embodiment of the present invention provides a pharmaceutical composition capable of delivering high doses of pyridoxal 5′-phosphate. Prior art formulations currently available, generally deliver up to 50 mg of pyridoxal 5′-phosphate per dosage form. Accordingly, the prior art formulations must be administered two, three or more times per day to achieve the desired therapeutic levels of pyridoxal 5′-phosphate. In contrast, a pharmaceutical composition of the present invention has a high proportion of pyridoxal 5′-phosphate or a pharmaceutically acceptable salt thereof.

An individual dosage form of the pharmaceutical compositions may contain between 250 and 1000 mg of pyridoxal 5′-phosphate. Pharmaceutical compositions according to the invention are suitable for once or twice daily administration.

A high proportion of pyridoxal 5′-phosphate or its salt allows a pharmaceutical composition to be provided in a dosage form that is smaller in size than the dosage forms of prior art formulations. Thus, a pharmaceutical composition according to the invention is easy to administer and is especially useful for patients who find it difficult to swallow large tablets or capsules.

Formulations

The present invention includes a pyridoxal-5′-phosphate pharmaceutical formulation suitable for oral administration comprising a dissolution profile, when measured in a standard dissolution apparatus, according to the United States Pharmacopoeia dissolution test, at 37° C. in a 0.05M phosphate buffered solution having a pH of 6.8 at 75 rpm, as follows: (a) greater than about 30% at 15 minutes, (b) greater than about 85% at 30 minutes, (c), greater than about 90% at 45 minutes, or (d) greater than about 95% at 60 minutes. Additionally, in vivo oral intake of between 15 and 60 mg/kg of an embodiment of the pyridoxal-5′-phosphate pharmaceutical formulation can produce a maximum plasma level (Cₘₐₓ) of between about 1 mg/L and 8 mg/L. The structural integrity of coated embodiments of the pharmaceutical compositions of the invention is minimally affected by acidic conditions of the stomach. Thus, an embodiment of the pharmaceutical compositions may have a dissolution profile of less than or equal to 10% dissolution at 120 minutes according to the United States Pharmacopoeia dissolution test in 0.1N HCl at 37° C. at 75 rpm.

Pharmaceutical compositions according to the present invention provide improved pyridoxal-5′-phosphate bioavailability. In vivo oral intake of between 15 and 60 mg/kg of the composition can produce a maximum plasma level (Cₘₐₓ) of pyridoxal-5′-phosphate of between about 1 and about 8 mg/L. Preferably, in vivo oral intake of between 15 and 60 mg/kg of the composition produces an average plasma level of between about 0.1 to about 2 mg/L of pyridoxal 5′phosphate in the period from 2 hours after intake to 24 hours after intake.

In an embodiment, the pharmaceutical composition comprises about 66.3% w/w pyridoxal-5′-phosphate or a pharmaceutically acceptable salt thereof, about 21.6% w/w microcrystalline cellulose, about 4.0% croscarmellose sodium, about 4.7% w/w povidone, about 2.0% talc, about 0.5% w/w colloidal silicon dioxide, and about 1.0% w/w magnesium stearate.

A pharmaceutical composition according to the invention may be prepared using either pyridoxal 5′-phosphate or a pharmaceutically acceptable salt thereof. Both the monohydrate and the anhydrous forms of pyridoxal 5′-phosphate are suitable for preparation of the pharmaceutical compositions of the invention. The pyridoxal 5-phosphate may be provided as salt forms with pharmaceutically compatible counterions such as but not limited, to citrate, tartate, bisulfate, etc. The pharmaceutically compatible salts may be formed with many acids, including but, not limited to, hydrochloric, sulfuric, acetic, lactic, tartaric, malic, succinic, etc. The salt forms tend to be more soluble in aqueous or other protonic solvents than the corresponding free base forms.

Preferably, pharmaceutical composition comprises a microcrystalline having a particle size of about 0.100 mm such as but not limited to, Avicel PH102. The povidone preferably has a K value of 27 to 33. In a preferred embodiment, the povidone is PVP K30.

A pharmaceutical composition according to the invention may further comprise additional pharmaceutically acceptable carriers, dispersants and excipients. Suitable excipients include fillers such as sugars, including lactose, sucrose, mannitol, or sorbitol, or cellulose preparations such as, maize starch, wheat starch, rice starch, potato starch, gelatin, gum tragacanth, methyl cellulose, hydroxypropylmethyl-cellulose, sodium carboxymethylcellulose, and/or polyvinylpyrrolidone. Disintegrating agents may include cross-linked polyvinylpyrrolidone, agar, or alginic acid or a salt thereof such as sodium alginate. The pharmaceutical composition also may comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols. Further excipients may comprise anti-adhesives such as talc, colloidal silicon dioxide, titanium dioxide, calcite, microcrystalline cellulose, metallic stearates, and barium sulphates. The composition can also include a granulation binder such as, but limited to, alginic acid.

In an embodiment of the invention, a pharmaceutical composition comprises: (a) a core, wherein said core comprises pyridoxal-5′-phosphate or a pharmaceutically acceptable salt thereof; (b) a sub-coat surrounding the core; and (c) an enteric coat surrounding the sub-coat. Optionally, the pharmaceutical composition further comprises a cosmetic color coat surrounding the enteric coat.

In a further embodiment of the invention, the core comprises pyridoxal-5′-phosphate or pharmaceutically acceptable salt thereof, microcrystalline cellulose, croscarmellose sodium, povidone, talc, colloidal silicon dioxide, and magnesium stearate.

The sub-coat, (or sealing coat) and the enteric coat ensure that the core containing the pyridoxal 5′-phosphate is able to pass through the stomach intact and be selectively absorbed in the intestine. The enteric coat is pH dependent and is preferentially labile in the relatively alkaline conditions of the intestine as opposed to the acidic conditions of the stomach. The sub-coat or sealing coat provides additional protection to the core to ensure minimal disintegration of the core in the stomach. Sealing and enteric coats are well known in the art. Any suitable combination of sealing and enteric coats can be used to prepare the pharmaceutical compositions according to the invention so long as dissolution of the pyridoxal 5′-phosphate core is preferably limited to the intestine.

A sub-coat or sealing coat protects the tablet ingredients from the water in the aqueous enteric coating dispersion to assure the stability of the dosage form. The sub-coat comprises a resin such as shellac, zein, and the like and is applied to the dosage form by well known methods. Sub-coats used in sugar coating processes usually consist of alcoholic solutions (approximately 10-30% solids) of resins such as shellac, zein, cellulose acetate phthalate, or polyvinyl acetate phthalate. Shellac is preferably used in the form of a shellac-based formulation containing polyvinylpyrrolidone. Other suitable polymeric solutions can be used as a sub-coat, such as Opadry® IR-7000 White or a copolymer of dimethylaminoethyl methacrylate and methacrylic acid ester (Eudragit®).

Materials useful for preparing enteric coatings for pharmaceuticals are well-known. These most commonly are pH-sensitive materials which are relatively insoluble and impermeable at the pH of the stomach, but which are more soluble and permeable at the pH of the small intestine and colon. Any coating material which modifies the release of the active ingredient in the desired manner may be used. In particular, coating materials suitable for use in the practice of the invention include but are not limited to polymer coating materials, such as cellulose acetate phthalate, cellulose acetate trimaletate, hydroxypropyl methylcellulose phthalate, polyvinyl acetate phthalate, ammonio methacrylate copolymers such as Eudragit® RS and RL, poly acrylic acid and poly acrylate and methacrylate copolymers such as Eudragit® S and L, polyvinyl acetaldiethylamino acetate, hydroxypropyl methylcellulose acetate succinate, shellac; hydrogels and gel-forming materials, such as carboxyvinyl polymers, sodium alginate, sodium carmellose, calcium carmellose, sodium carboxymethyl starch, polyvinyl alcohol, hydroxyethyl cellulose, methyl cellulose, gelatin, starch, and cellulose based cross-linked polymers in which the degree of crosslinking is low so as to facilitate adsorption of water and expansion of the polymer matrix, hydoxypropyl cellulose, hydroxypropyl methylcellulose, polyvinylpyrrolidone, crosslinked starch, microcrystalline cellulose, chitin, aminoacryl-methacrylate copolymer (Eudragit® RS-PM), pullulan, collagen, casein, agar, gum arabic, sodium carboxymethyl cellulose, polyvinylpyrrolidone, anionic and cationic hydrogels, polyvinyl alcohol having a low acetate residual, a swellable mixture of agar and carboxymethyl cellulose, copolymers of maleic anhydride and styrene, ethylene, propylene or isobutylene, pectin, polysaccharides such as agar, acacia, karaya, tragacanth, algins and guar, polyacrylamides, polyethylene oxides, diesters of polyglucan, crosslinked polyvinyl alcohol and poly N-vinyl-2-pyrrolidone, sodium starch glucolate; hydrophilic polymers such as polysaccharides, methyl cellulose, sodium or calcium carboxymethyl cellulose, hydroxypropyl methyl cellulose, hydroxypropyl cellulose, hydroxyethyl cellulose, nitro cellulose, carboxymethyl cellulose, cellulose ethers, polyethylene oxides, methyl ethyl cellulose, ethylhydroxy ethylcellulose, cellulose acetate, cellulose butyrate, cellulose propionate, gelatin, collagen, starch, maltodextin, pullulan, polyvinyl pyrrolidone, polyvinyl alcohol, polyvinyl acetate, glycerol fatty acid esters, polyacrylamide, polyacrylic acid, copolymers of methacrylic acid or methacrylic acid (e.g. Eudragit®), other acrylic acid derivatives, sorbitan esters, natural gums, lecithins, pectin, alginates, ammonia alginate, sodium, calcium, potassium alginates, propylene glycol alginate, agar, and gums such as arabic, karaya, locust bean, tragacanth, carrageens, guar, xanthan, scleroglucan and mixtures and blends thereof. The thickness of the coating is adjusted to give the desired delay property. In general, thicker coatings are more resistant to erosion and, consequently, yield a longer delay.

In an embodiment of the invention, a sub-coat is Opadry® IR-7000 White (Colorcon, West Point, Pa.) and constitutes about 2.0% to about 5.0% w/w of the total composition. An enteric coat is preferably Acryl-EZE White (Colorcon) and constitutes about 10-14% w/w, and more preferably about 10 % w/w of total composition. A color coat is preferably Opadry® Fx Blue (Colorcon) and constitutes about 1.0% to about 3.0% w/w, and more preferably about 1.5% w/w of the total composition.

In a preferred embodiment of the invention, the sub-coat or sealing coat is Opadryl IR-7000 White and constitutes about 3.0 % w/w of the total composition and the enteric coat is preferably Acryl-EZE White and constitutes about 10.0% w/w of total composition.

Absorption of the coated embodiments of the pharmaceutical compositions is preferentially limited to the intestine. The pharmaceutical compositions selectively and efficiently dissolve in the relatively alkaline environment of the intestine. The inventors have determined that the use of two distinct disintegrants, microcrystalline cellulose together with croscarmellose, promotes more rapid disintegration of the granules following disintegration of the oral dosage form, in the intestine. The use of about 11.9% w/w of a microcrystalline cellulose, preferably Avicel PH 102, and about 2.0% w/w of croscarmellolose results in faster dissolution of the composition and a more consistent rate of dissolution.

In a further embodiment, the present invention provides a pre-blend useful in the manufacture of a pyridoxal 5′-phosphate oral dosage form. Powdered preparations of pyridoxal-5′-phosphate suffer poor flowability. As a consequence, it is difficult to prepare tablets of pyridoxal 5′-phosphate in a consistent manner. Because powdered pyridoxal 5′-phosphate does not tend to disperse evenly, it is difficult to uniformly blend and granulate pyridoxal 5′-phosphate with other ingredients (i.e. exicipients) prior to tableting.

In tablets having a high concentration of pyridoxal 5′-phosphate, it may be necessary to granulate the pyridoxal 5′-phosphate in order to alter its physical properties into a material that can flow. Good flow properties are essential for tableting since the powder has to be able to flow into the die cavity in which the tablet will be formed with punches. If the powder does not flow evenly and quickly, it is difficult to control tablet weights. Poor flow properties also necessitates the use of very slow compression speeds which are impractical for commercial purposes.

An embodiment of the invention provides a pre-blend for the manufacture of a pyridoxal-5′-phosphate oral dosage form comprising at least about 50% w/w pyridoxal-5′-phosphate or a pharmaceutically acceptable salt thereof

A specific embodiment of the invention provides a pre-blend for the manufacture of a pyridoxal-5′-phosphate oral dosage form comprising: about 82.7% w/w pyridoxal 5′-phosphate, about 14.8% w/w microcrystalline cellulose, and about 2.5% w/w croscarmellose sodium. The flow characteristics of the pre-blend, as compared to powdered pyridoxal 5′-phosphate alone, allows for improved ease in handling and in blending of the active ingredient with other ingredients such as but not limited to disintegrants, binding agents, lubricants.

A pre-blend can be prepared by blending pyridoxal-5′-phosphate, microcrystalline cellulose, and croscarmellose sodium in a high shear mixer.

In a further embodiment, the invention provides a method of preparing an enteric coated oral dosage form of pyridoxal-5′-phosphate. A method comprises a first step of dissolving a granulation binder, such as povidone, in purified water to provide a granulating solution. In a specific embodiment, a method comprises the first step of dissolving about 4.7% w/w of povidone in purified water to provide a granulating solution. The povidone is preferably a povidone having a K value of between 27 and 30 and is more preferably PVP K30.

A pyridoxal-5′-phosphate containing pre-blend is prepared by mixing about 50% w/w pyridoxal-5′-phosphate powder or a pharmaceutically acceptable salt thereof with disintegrants or mixtures of disintegrants, such as for example, croscarmellose sodium. In a specific embodiment, a pyridoxal 5′-phosphate containing pre-blend is prepared by mixing about 66.3 % w/w pyridoxal-5′-phosphate (or a pharmaceutically acceptable salt thereof) powder with about 11.9% w/w microcrystalline cellulose and about 2.0% w/w of croscarmellose sodium. Microcrystalline cellulose may preferably be a microcrystalline cellulose having a particle size of about 0.100 mm, and more preferably the microcrystalline cellulose is Avicel PH102. A pre-blend is preferably prepared using a high shear mixer. Use of a high shear mixer results in improved initial blending and granulation. Use of a high shear mixer significantly reduces problems associated with the use of other types of mixers such as ribbon blenders. In particular, high shear mixers, as compared to ribbon blenders, do not have “dead spots” and as such provides more uniform blending. Use of the high shear mixer provides better control and consistency during granulation. In addition high shear mixers are easier to clean between batches as compared to ribbon blenders. Accordingly, the incidence of contamination and variation between batches is significantly reduced with the use of a high shear mixer.

A pre-blend can be mixed with a granulating solution to provide a granulating preparation. Preferably the granulating solution and the pre-blend are combined by spraying the granulating solution into a high shear mixer as the pre-blend is being mixed. In a further preferred embodiment, a wet granulating preparation is sized using a conical mill with a 0.5″ screen. A resulting granulating preparation is then dried to a moisture content of about 1.5% as determined by loss on drying (LOD) testing, using a fluid bed dryer set at 60° C. It is advantageous to use a fluid bed dryer rather than other drying systems such as forced air ovens. Use of a fluid bed dryer allows the granulating preparation to be dried quickly and uniformly. Once a granulating preparation is dried, the preparation is sized using an oscillating granulator fitted with 20 mesh screen. Use of finer screens during the initial granulation steps produces smaller granules which facilitate granule disintegration in vivo. In addition, screening yields a more uniform blend having particles of consistent size. A sized granulating preparation is then blended using a diffusive blender prior to the addition of further disintegrants, binding agents, lubricants, glidants, and anti-adherents. Preliminary blending of the dried and sized granules provides a convenient and uniform sample for the determination of moisture content by LOD testing or Karl Fischer (KF) testing.

An exicipient preparation is prepared by combining further excipients, such as microcrystalline cellulose, croscarmellose sodium, talc, and colloidal silicon dioxide using a diffusive blender. In a specific embodiment, an exicipient preparation is provided by combining about 9.7% w/w microcrystalline cellulose, about 2.0% w/w croscarmellose sodium, about 2.0% w/w talc, and about 0.5% w/w colloidal silicon dioxide using a diffusive blender. Colloidal silicon dioxide is extremely fine and readily forms agglomerates. By mixing colloidal silicon dioxide with the microcrystalline cellulose, croscarmellose sodium, and talc, colloidal silicon dioxide is densified thereby facilitating screening and preventing re-agglomeration. A mixture of microcrystalline cellulose, croscarmellose sodium, talc, and colloidal silicon dioxide can be first passed through a 20 mesh screen and then thoroughly mixed using a diffusive blender. A resulting excipient preparation can be then sized again using an oscillating granulator fitted with a 20 mesh screen to break up any agglomerates.

A dried, sized, and blended granulating preparation and a sized excipient preparation can then be mixed together using a diffusive blender to provide a semi-final blend preparation. In a specific embodiment, a dried, sized and blended granulating preparation and the sized excipient preparation are then mixed together using a diffusive blender to provide a semi-final blend preparation.

A lubricant, such as magnesium stearate, can be sized by passing it through a 30 mesh screen. A sized lubricant can then be blended with a semi-final blend preparation using a diffusive blender to provide a final blend. Overmixing a lubricant with other components of a composition should be avoided. A lubricant and a semi-final blend preparation can generally be mixed together for about 3 to 5 minutes to provide a preparation, which avoids overmixing. Overmixing a lubricant produces oral dosage forms having many problems including retarded dissolution. Addition of magnesium stearate at the end of the blending yields oral dosage forms having preferred dissolution profiles. Preferably, a lubricant, such as magnesium sterate, is the last component added to the pharmaceutical preparation to avoid overmixing.

In a specific embodiment, about 1.0% w/w magnesium stearate is sized by passing it through a 30 mesh screen. Sized magnesium stearate is then blended with the semi-final blend preparation using a diffusive blender. Magnesium stearate and the semi-final blend preparation are generally mixed together for about 3 to 5 minutes to provide a final tableting preparation.

A tableting preparation can then be compressed into a core using conventional methods and apparatus known in the art.

A sub-coat (or sealing coat) can be applied to a core to provide a sub-coated core (or sealed core). A sub-coated core (or sealed core) can then be coated with an enteric coating.

In a preferred embodiment, a sub-coat or sealing coat is applied as a 5% w/w dispersion of Opadryl-IR-7000 White. An enteric coat is then applied to the sealed core. Preferably, the enteric coat is applied as a 15% w/w dispersion of Acryl EZE White. Acryl EZE White provides superior enteric coating properties compared to other enteric coating systems such as Sureteric YAE-6-18107 White. Use of Acryl EZE White is also advantageous as it is easier to use and handle as compared to other enteric coating systems. A side vented perforated coating pan or other suitable device can be used to apply the coatings by conventional methods.

The following are specific embodiments of the invention:

In an embodiment of the invention, the composition has a dissolution profile of greater than about 30% at 15 minutes according to the United States Pharmacopoeia dissolution test in a 0.05M phosphate buffered solution having a pH of 6.8.

In an embodiment of the invention, the composition has a dissolution profile of greater than about 85% at 30 minutes according to the United States Pharmacopoeia dissolution test in a 0.05M phosphate buffered solution having a pH of 6.8.

In an embodiment of the invention, the composition has a dissolution profile of greater than about 90% at 45 minutes according to the United States Pharmacopoeia dissolution test in a 0.05M phosphate buffered solution having a pH of 6.8.

In an embodiment of the invention, the composition has a dissolution profile of greater than about 95% at 60 minutes according to the United States Pharmacopoeia dissolution test in a 0.05M phosphate buffered solution having a pH of 6.8.

In an embodiment of the invention, the composition has a dissolution profile up to 10% dissolution in 120 minutes in 0.1N HCl at 37° C. at 75 rpm, according to the United States Pharmacopoeia dissolution test.

In an embodiment of the invention, in vivo oral intake of between 15 and 60 mg/kg of the composition produces of maximum plasma level (Cₘₐₓ) of pyridoxal-5′-phosphate of between about 1 and about 8 mg/L from 2 hours after intake to 24 hours after intake.

In an embodiment of the invention, in vivo oral intake of between 15 and 60 mg/kg of the composition produces an average plasma level of between about 0.1 and about 2 mg/L of pyridoxal-5′-phosphate in the period from 2 hours after intake to 24 hours after intake.

In an embodiment of the invention, the pharmaceutical composition comprises: (a) a core, wherein said core comprises the pyridoxal-5′-phosphate or pharmaceutically acceptable salt thereof; (b) a sub-coat surrounding the core; and (c) an enteric coat surrounding the sub-coat.

In an embodiment of the invention, the core further comprises a disintegrant or mixture of disintegrants. The disintegrant can be microcrystalline cellulose, croscarmellose sodium, or a mixture thereof.

In an embodiment of the invention, the core further comprises a granulation binder. In a further embodiment of the invention, the granulation binder is povidone with a K value of between 27-33. In an embodiment of the invention, povidone is PVP K-30.

In an embodiment of the invention, the microcrystalline cellulose has a particle size of about 0.100 mm.

In an embodiment of the invention, the microcrystalline cellulose is Avicel® PH 102.

In an embodiment of the invention, the sub-coat or sealing coat is Opadry®-IR-7000 White.

In an embodiment of the invention, the amount of Opadry®-IR-7000 White is about 3% w/w

In an embodiment of the invention, the enteric coat is Acryl-EZE White.

In an embodiment of the invention, the amount of Acryl-EZE White is about 10% w/w.

In an embodiment, a pharmaceutical composition for oral administration comprises: about 65% to 75% w/w pyridoxal-5′-phosphate or a pharmaceutically acceptable salt thereof, about 20% to about 30% w/w microcrystalline cellulose, about 2.0% to about 4.0% croscarmellose sodium, about 3.0% to about 6.0% w/w povidone, about 1.0% to about 4.0% talc, about 0.1% to about 1.0% w/w colloidal silicon dioxide, and about 0.5% to about 1.5% w/w magnesium stearate.

In an embodiment, the present invention provides a pre-blend for the manufacture of a pyridoxal-5′-phosphate oral dosage form comprising about 66.3% w/w pyridoxal 5′-phosphate.

An embodiment provides a method of preparing an oral dosage form of pyridoxal-5′-phosphate comprising the steps of:
(a) dissolving a granulation binder in purified water to provide a granulating solution;
(b) mixing at least 50% w/w/ pyridoxal-5′-phosphate or pharmaceutically acceptable salt with a disintegrant or a mixture of disintegrants to provide a pre-blend;
(c) mixing the pre-blend with the granulating solution to provide a granulating preparation;
(d) substantially drying the granulating preparation;
(e) mixing excipients with the granulating preparation to provide a semi-final blend preparation;
(f) mixing the semi-final blend preparation with a lubricant to provide a final blend preparation;
(g) compressing the final blend preparation into a core;
(h) applying a sub-coat to the core to provide a sub-coated core; and
(i) applying an enteric coat to the sub-coated core.

In an embodiment, the present invention provides a method of preparing a an oral dosage form of pyridoxal-5′-phosphate comprising the steps of: (a) dissolving about 3.0% to about 6.0% w/w povidone in purified water to provide a granulating solution; (b) mixing about 65% to about 75% w/w pyridoxal-5′-phosphate or a pharmaceutically acceptable salt thereof with about 8.0% to about 16.0% w/w microcrystalline cellulose and about 1.0% to about 3.0% w/w croscarmellose sodium to provide a pre-blend; (c) mixing the pre-blend with the granulating solution to provide a granulating preparation; (d) substantially drying the granulating preparation; (e) mixing about 7.0% to about 14.0% w/w microcrystalline cellulose, about 1.0% to about 3.0% w/w croscarmellose sodium, about 1.0% to about 4.0% w/w talc, and about 0.1% to about 1.0% w/w colloidal silicon dioxide, to provide an exicipient preparation; (f) mixing the granulating preparation with the exicipient preparation to provide a semi-final blend preparation; (g) mixing the semi-final blend preparation with about 0.5% to about 1.5% w/w magnesium stearate to provide a final tableting preparation; (h) compressing the tableting preparation into a core; (i) applying a sub-coat to the core to provide a sub-coated core; and 0) applying an enteric coat to the sub-coated core and, optionally, (k) applying a color coat to the enteric coated tablets

In an embodiment of the invention, the sub-coat is an about 15% w/v dispersion of Opadry®l-IR-7000 White applied to a about 2.0% to about 5.0% weight gain to the tablet core.

In an embodiment of the invention, the enteric coat is an about 20% w/v dispersion of Acryl-EZE White applied to an about 8.0 to about 14.0% weight gain to the sub coated tablet core.

In an embodiment of the invention, the color coat is a 7.5% w/v dispersion of Opadry® Blue Fx applied to an about 1.0% to about 3.0% weight gain to the enteric coated tablet core.

In an embodiment of the invention, the pyridoxal-5′-phosphate, the microcrystalline cellulose, and the croscarmellose are mixed with a high shear mixer to provide a pre-blend.

In an embodiment of the invention, the pre-blend and the granulating solution are mixed by spraying the granulating solution onto the pre-blend while the pre-blend is being mixed in the high shear mixer.

In an embodiment of the invention, the method also comprises a further step of passing the granulating preparation through a conical mill with a 0.5″ screen following step (c) and prior to step (d).

In an embodiment of the invention, the granulating preparation is dried using a fluid bed dryer set to an inlet temp of 60° C.

In an embodiment of the invention, the method comprises the further step of passing the granulating preparation through a 20 mesh screen and then mixing the granulating preparation in a diffusive blender, following step (d) and prior to step (e).

In an embodiment of the invention, the method comprises further steps of mixing the pre-blend preparation using a small diffusive blender and passing the mixed pre-blend preparation through a 20 mesh screen.

In an embodiment of the invention, the granulating preparations and pre-blend preparations are mixed using a diffusive blender.

In an embodiment of the invention, the method comprises a further step of passing magnesium stearate through a 30 mesh screen prior to mixing magnesium stearate with a semi-final blend preparation.

In an embodiment of the invention, a semi-final blend preparation and magnesium stearate are mixed using a diffusive blender.

A composition according to embodiments of the pyridoxal-5′-phosphate formulations may be incorporated into any suitable dosage form which facilitates its release. Unit cores as described herein can be formulated as a particulate. A multiparticulate composition can be filled into suitable capsules, such as hard or soft gelatin capsules. Alternatively, a multiparticulate composition may be compressed (optionally with additional excipients) into mini-tablets that can be subsequently filled into capsules. Another suitable dosage form is a tablet, wherein the particulates are compressed into tablet form. Pyridoxal-5′-phosphate containing particles making up a composition may further be included in rapidly dissolving dosage forms such as an effervescent dosage form or a fast-melt dosage form.

Methods of Treatment

A further embodiment of, the present invention provides a method of reducing the incidence of nausea and vomiting associated with oral administration of pyridoxal 5′-phosphate or a pharmaceutically acceptable salt thereof, said method comprising a step of administering an effective amount of pyridoxal 5′-phosphate in a controlled release, delayed release, or a combination of a controlled release and delayed released oral pharmaceutical composition.

“Controlled release” refers to any formulation technique wherein release of the active substance from the dosage from is modified to occur at a slower rater than that from an immediate release product, such as a conventional swallow tablet or capsule.

By delayed release is meant any formulation technique wherein release of the active substance from the dosage form is modified to occur at a later time than that from a conventional immediate release product. Subsequent release of active substance from a delayed release formulation may also be controlled as defined above.

Such controlled release formulations are preferably formulated in a manner such that release of the pyridoxal 5 ′-phosphate is effected predominantly during the passage through the stomach and the small intestine, and delayed release formulations are preferably formulated such that release of active substance is avoided in the stomach and is effected predominantly during passage through the small intestine. The small intestine is suitably the duodenum, the ileum or the jejunem.

In a preferred embodiment, the controlled release or delayed release pharmaceutical composition is a pharmaceutical composition according to the invention, comprising about 66.3% w/w pyridoxal-5′-phosphate or a pharmaceutically acceptable salt thereof, about 21.6% w/w microcrystalline cellulose, about 4.0% w/w croscarmellose sodium, about 4.7% w/w povidone, about 2.0% w/w talc, about 0.5% w/w colloidal silicon dioxide, and about 1.0% w/w magnesium stearate, wherein the composition is in the form of a tablet comprising: (a) a core, wherein said core comprises the pyridoxal-5′-phosphate, the microcrystalline cellulose, the croscarmellose sodium, the povidone, the talc, the colloidal silicon dioxide, and the magnesium stearate; (b) a sub-coat surrounding the core; and (c) an enteric coat surrounding the seating coat. Any of the coated pharmaceutical compositions of the invention can be used to reduce the incidence of nausea and vomiting associated with the oral administration of pyridoxal 5′-phosphate or a pharmaceutically acceptable salt thereof.

Pharmaceutical compositions are generally administered in an amount effective for treatment or prophylaxis of a specific indication or indications. Optimum dosage can be determined by standard methods for each treatment modality and indication, taking into account an indication, its severity, route of administration, complicating conditions and the like. A therapeutically effective dose further refers to that amount of the compound sufficient to result in amelioration of symptoms associated with such disorders. Techniques for formulation and administration of the compounds of the instant application may be found in “Remington's Pharmaceutical Sciences,” Mack Publishing Co., Easton, Pa., latest edition. For administration to mammals, and particularly humans, it is expected that daily dosage levels of the active agent will be 100 to 1000 mg, typically around 500 mg. A physician may determine an actual dosage which will be most suitable for an individual and will vary with the age, weight, and response of the particular individual. The above dosages are exemplary of the average case. There can, of course, be individual instances where higher or lower dosage ranges are merited, and such are within the scope of this invention.

Although the invention has been described with reference to illustrative embodiments, it is to be understood that the invention is not limited to these precise embodiments, and that various changes and modifications may be effected therein by one skilled in the art. All such changes and modifications are intended to be encompassed in the appended claims.

### EXAMPLES

### Example One

### Pyridoxal 5′-phosphate Enteric Coated Tablet Formulation and Method of Preparation

Table 1 illustrates the ingredients and relative amounts for the preparation of enteric coated tablets of pyridoxal 5′-phosphate (265 mg per tablet). As set out in Table 1, one batch yields 20,000 tablets. The batch size can be scaled up or down by increasing or decreasing the relative amounts proportionately.

TABLE 1
Formulation for Enteric Coated Pyridoxal 5′-phosphate Tablets

| | | mg/ | g/ |
|---|---|---|---|
| Ingredient | % | tablet | batch |
| Granulation Phase | | | |
| Pyridoxal 5′-phosphate Powder | 66.3 | 265 | 5300 |
| Microcrystalline Cellulose (Avicel PH102) | 11.9 | 47.5 | 950 |
| Croscarmellose Sodium | 2.0 | 8 | 160 |
| Povidone (K-30) | 4.7 | 18.75 | 375 |
| Sub-Total: | 84.8 | 339.25 | 6785 |
| Purified Water (for PVP granulation solution) | | qs | 1500 |
| Additional Purified Water (for granulation) | | qs | 150 |
| Tableting Phase | | | |
| Granulation | 84.8 | 339.25 | 6785 |
| Microcrystalline Cellulose (Avicel PH102) | 9.7 | 38.75 | 775 |
| Croscarmellose Sodium | 2.0 | 8 | 160 |
| Talc | 2.0 | 8 | 160 |
| Colloidal Silicon Dioxide | 0.5 | 2 | 40 |
| Magnesium Stearate | 1.0 | 4 | 80 |
| Total: | 100.0 | 400 | 8000 |
| Coating | | | |
| Opadry-IR-7000 White (Seal Coat)-5% dispersion | 3.1 | 14.1 | 282 |
| Acryl-EZE White- (Enteric Coat)-15% | 10.2 | 47.1 | 942 |
| Coated Tablet Total: | 100.0 | 461.2 | |
| Purified Water (for Seal Coat) | | qs | 1598 |
| Purified Water (for Enteric Coat) | | qs | 5338 |

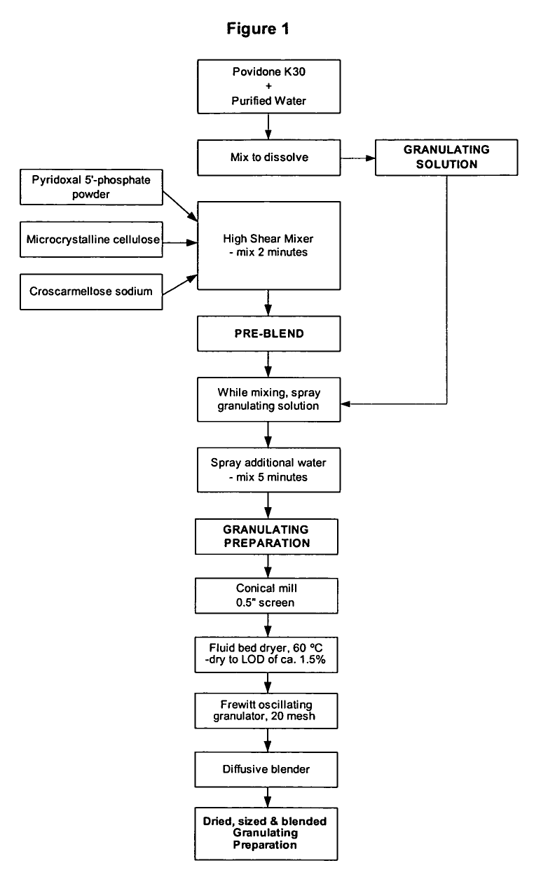

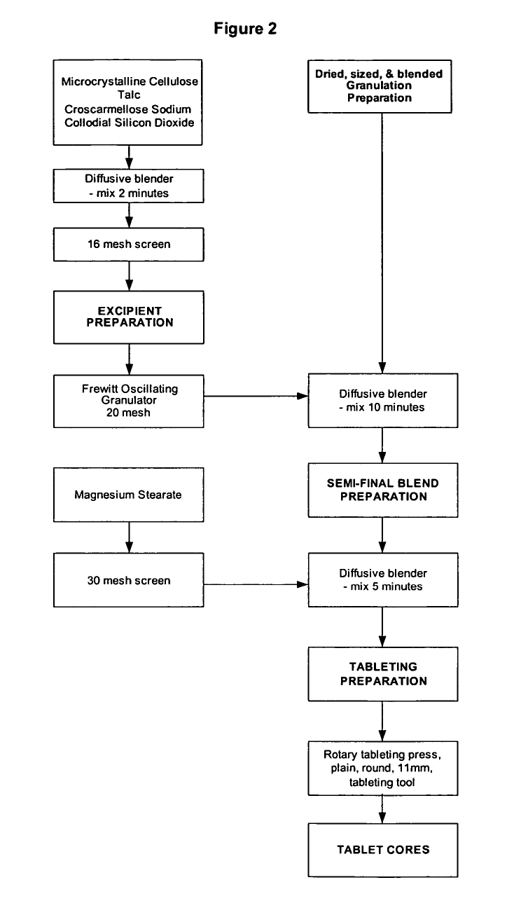

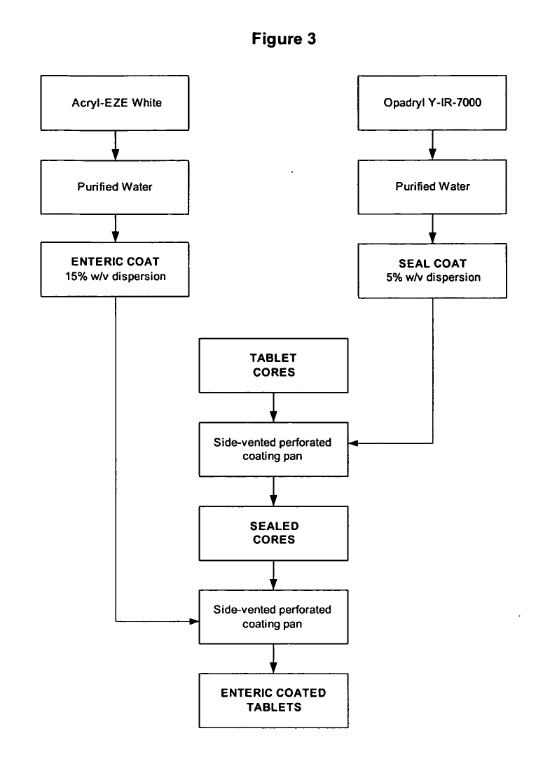

The pyridoxal 5′-phosphate enteric coated tablets were prepared in a three step process: (1) granulation phase, (2) tableting phase, and (3) coating phase. FIGS. 1 to 3 illustrate the steps involved in preparing the tablets.

Granulation and Blending Phase—A granulating solution was prepared by dissolving the Povidone K30 in a suitable amount of purified water. A pyridoxal 5′phosphate pre-blend was prepared by mixing the pyridoxal 5′-phosphate powder with the first amount of microcrystalline cellulose (Avicel PH 102), and the first amount of croscarmellose sodium for approximately 2 minutes in a high shear mixer. While continuing to mix the pre-blend, the granulating solution was sprayed into the mixer. Additional water was added as necessary for the granulating process. The pre-blend and the granulation solution were mixed for approximately 5 minutes to provide a granulating preparation. The resulting granules were sized by passing the granules through a conical mill (Comil) with a 0.5″ screen. The sized granules were then dried in a fluid bed dryer at 60° C. until the granules have a LOD of approximately 1.5%. The dried granules were sized using a Frewitt oscillating granulator with a 20 mesh screen. The sized granules were then blended using a diffusive blender for about 5 minutes. The second amount of microcrystalline cellulose (Avicel PH 102), the second amount of croscarmellose sodium, the talc, and the colloidal silicon dioxide were mixed for approximately 2 minutes using a diffusive blender to provide an exicipient preparation. The excipient preparation was combined with the dried, sized, and blended granulation preparation and mixed in a diffusive blender for approximately 10 minutes to provide a semi-final blend preparation. The magnesium stearate was sized using a 30 mesh screen and blended with the semi-final blend preparation for approximately 5 minutes using a diffusive blender to provide the final tableting preparation.

Tableting phase—The tableting preparation was compressed into cores using a rotary tablet press and a plain, 11 mm, round, standard, concave tablet tool.

Coating phase—The sealing coat was prepared by dispersing the Opadryl Y-IR-7000 in a suitable about of purified water to provide a 5% w/v dispersion. A sufficient amount of the Opadryl Y-IR-7000 dispersion was applied such that amount of applied Opadryl Y-IR-7000 was about 3.1% w/w relative to the total weight to the finished tablet. The enteric coating was prepared by dispersing the Acryl-EZE White in a suitable amount of purified water to provide a 15% w/v dispersion. A sufficient amount of the Acryl-EZE White was applied such that the amount of Acryl-EZE White was about 10.2% w/w relative to the total weight of the finished tablet. Using a side vented perforated coating pan, the tablet cores were first coated with the sealing coat dispersion. The tablets were then coated with the enteric coat dispersion.

### Example Two

### Dissolution Studies for Pyridoxal 5′-phosphate Enteric Coated Tablets and Uncoated Tablet Core

The dissolution properties of the pyridoxal 5 ′-phosphate enteric coated tablets and uncoated tablet cores were determined using conventional testing methods. The dissolution test was performed in a VanKel Model Vanderkamp 600 (6 spindle) dissolution apparatus equipped with an autosampler, digital thermometer and timer. A paddle speed was set up at 75 rpm. The sampling volume was 10 ml. A 2-stage dissolution procedure was carried out based on USP <724> method B for enteric coated tablets. The Acid Stage was carried out using 0.1N HCl for 120 minutes at 37° C. followed by the buffer stage at pH 6.8 at 37° C.

The dissolution data for the enteric coated tablets were observed within the following specification limits:
dissolution in a 0.05M phosphate buffered solution having a pH of 6.8 of greater than 60% at 30 minutes;
dissolution in a 0.05M phosphate buffered solution having a pH of 6.8 of greater than 80% at 60 minutes; and
dissolution in a 0.1N HCl at 120 minutes, not more than=10%

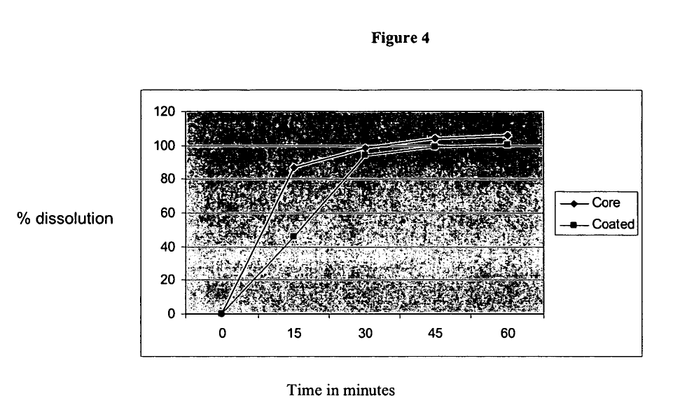

FIGS. 4 to 7 are line graphs illustrating the dissolution profile of the enteric coated tablets versus dissolution of the core of the tablet, demonstrating the delayed dissolution of the tablets when coated by the methods of the invention.

FIG. 4 illustrates the dissolution profiles for enteric coated cores and tablets prepared using a low rate of granulating solution application (“Granulation 1”). The granulating preparation was dried to a moisture content of about 1.5% prior to tableting.

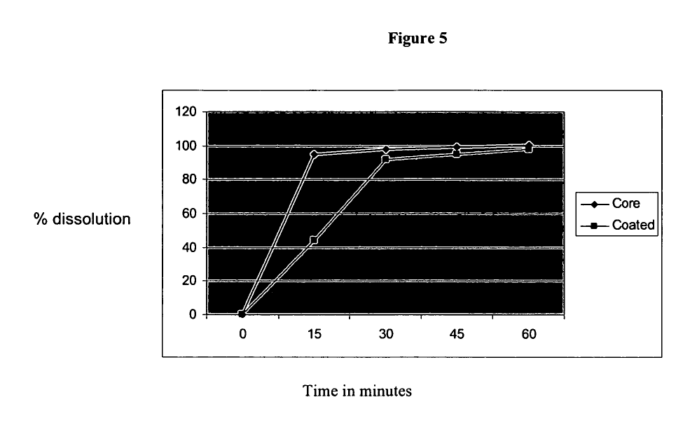

FIG. 5 illustrates the dissolution profiles for enteric coated cores and tablets prepared using a high rate of granulating solution application (“Granulation 2”). The granulating preparation was dried to a moisture content of about 1.5% prior to tableting.

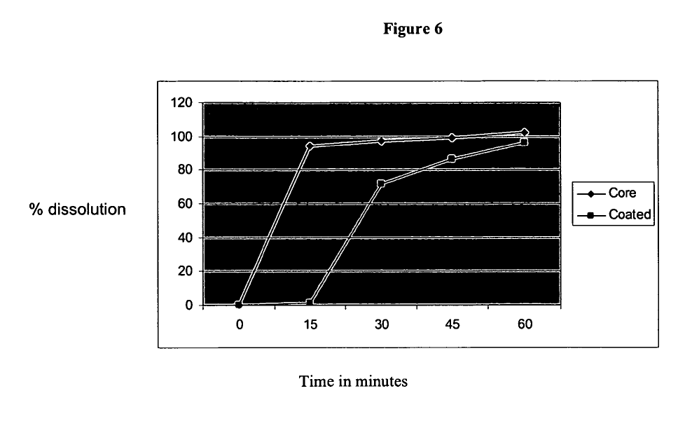

FIG. 6 illustrates the dissolution profiles for enteric coated cores and tablets prepared using a mid rate of granulating solution application (“Granulation 3”). The granulating preparation was dried to a moisture content of about 1.0% prior to tableting.

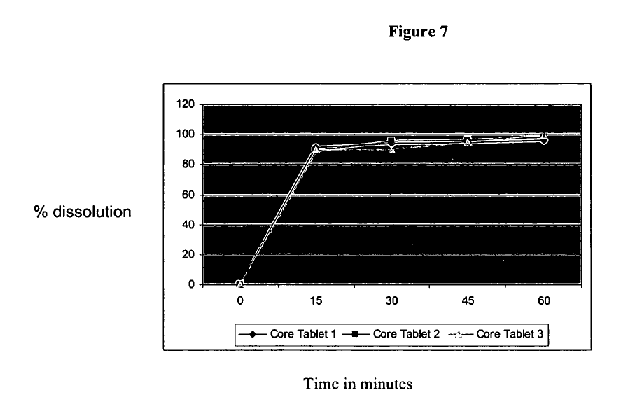

The individual dissolution profiles of Granulation 1, 2, and 3 are compared in FIG. 7.

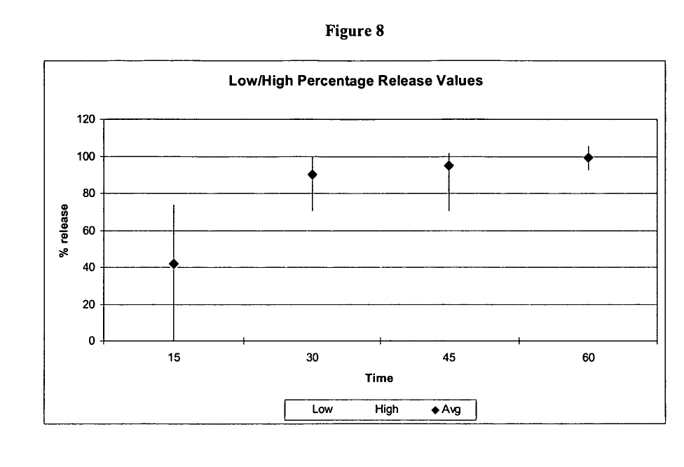

FIG. 8 illustrates the low, high and average percentage release values for Granulation 1, 2, and 3.

### Example Three

### Dissolution Studies for Pyridoxal 5′-phosphate Enteric Coated Tablets

The dissolution properties of the 250 mg pyridoxal 5′-phosphate enteric coated tablets were determined using conventional testing methods.

Disintegration time was determined using USP method <701> in simulated gastric fluid (minus pepsin) and in simulated intestinal fluid (minus pancreatin).

The tablets remained intact after 1 hour in the simulated gastric fluid. Complete disintegration of the tablets in the simulated intestinal fluid was observed at between 5:46 to 14:52 minutes.

Dissolution time was determined using USP <711> and USP <724.> method B for enteric coated tablets. The paddle speed of the dissolution apparatus was set at 100 rpm with sampling points at 30 and 45 minutes. The Concentration of the pyridoxal 5′-phosphate in the dissolution buffers was determined by LCMS

The dissolution data for the enteric coated tablets were observed within the following specification limits:
dissolution in a 0.05M phosphate buffered solution having a pH of 6.8 of greater than 80% at 45 minutes; and
dissolution in a 0.1N HCl at 120 minutes, not more than=10%

The tablets remained intact following 120 minutes in 0.1N HCl. After 30 minutes in pH 6.8 buffer, the observed dissolution was between 91.5 and 93.3%. After 45 minutes in the pH 6.8 buffer, the observed dissolution was between 92.5 and 100%.

### Example Four

### Safety, Tolerance and Pharmacokinetics Study of Pyridoxal 5′-phosphate Enteric Coated Tablets

A single center, Phase I, open label study was conducted to evaluate the safety, tolerance, and pharmacokinetics of pyridoxal 5′-phosphate (p5p) enteric coated tablets.

Subjects—Each study cohort consisted of 6 subjects (3 males, 3 females) and included:
Male or female, smoker or non-smoker, ≧18 and ≦55 years of age,
Capable of consent, and
BMI ≧9.0 and <30.0 kg/m2.

Subjects to whom any of the following applies were excluded from the study:
Clinically significant illnesses within 4 weeks prior to the administration of the study medication;
Clinically significant surgery within 4 weeks prior to the administration of the study medication;
Any clinically significant abnormality found during medical screening;
Any reason which, in the opinion of the Medical Sub-Investigator, would prevent the subject from participating in the study;
Abnormal laboratory tests judged clinically significant;
Positive testing for hepatitis B, hepatitis C, or HIV at screening;
ECG abnormalities (clinically significant) or vital sign abnormalities (systolic blood pressure lower than 100 or over 140 mmHg, diastolic blood pressure lower than 60 or over 90 mmHg, or heart rate less than 60 or over 100 bpm) at screening;
History of significant alcohol abuse or drug abuse within one year prior to the screening visit;
Regular use of alcohol within six months prior to the screening visit (more than fourteen units of alcohol per week [1 Unit=150 mL of wine, 360 mL of beer, or 45 mL of 40% alcohol]);
Use of soft drugs (such as marijuana) within 3 months prior to the screening visit or hard drugs (such as cocaine, phencyclidine [PCP] and crack) within 1 year prior to the screening visit or positive urine drug screen at screening;
History of allergic reactions to heparin, pyridoxal-5′-phosphate, vitamin B6, other pyridoxines, or other related drugs;
Use of any drugs known to induce or inhibit hepatic drug metabolism (examples of inducers: barbiturates, carbamazepine, phenytoin, glucocorticoids, omeprazole; examples of inhibitors: antidepressants (SSRI), cimetidine, diltiazem, macrolides, imidazoles, neuroleptics, verapamil, fluoroquinolones, antihistamines) within 30 days prior to administration of the study medication;
Use of an investigational drug or participation in an investigational study within 30 days prior to administration of the study medication;
Clinically significant history or presence of any clinically significant gastrointestinal pathology (e.g. chronic diarrhea, inflammatory bowel diseases), unresolved gastrointestinal symptoms (e.g. diarrhea, vomiting), liver or kidney disease, or other conditions known to interfere with the absorption, distribution, metabolism, or excretion of the drug;
Any clinically significant history or presence of clinically significant neurological, endocrinal, cardiovascular, pulmonary, hematologic, immunologic, psychiatric, or metabolic disease;
Use of prescription medication within 14 days prior to administration of study medication or over-the-counter products (including natural food supplements, vitamins, garlic as a supplement) within 7 days prior to administration of study medication, except for topical products without systemic absorption or hormonal contraceptives;
Smoking more than 25 cigarettes per day;
Any food allergy, intolerance, restriction or special diet that, in the opinion of the Medical Sub-Investigator, could contraindicate the subject's participation in this study;
A depot injection or an implant of any drug (other than hormonal contraceptive) within 3 months prior to administration of study medication;
Donation of plasma (500 mL) within 7 days prior to drug administration. Donation or loss of whole blood (excluding the volume of blood that will be drawn during the screening procedures of this study) prior to administration of the study medication as follows:
   50 mL to 300 mL of whole blood within 30 days,
   301 mL to 500 mL of whole blood within 45 days, or
   more than 500 mL of whole blood within 56 days prior to drug administration;
Breast-feeding subject;
Positive urine pregnancy test at screening; and
Female subjects of childbearing potential having unprotected sexual intercourse with any non-sterile male partner (i.e. male who has not been sterilized by vasectomy for at least 6 months) within 14 days prior to study drug administration. Acceptable methods of contraception:
   intra-uterine contraceptive device (placed at least 4 weeks prior to study drug administration;
   condom or diaphragm+spermicide;
   hormonal contraceptives (starting at least 4 weeks prior to study drug administration).

Restrictions—Subjects were instructed to abstain from:
smoking from at least 2 hours prior to dosing until 6 hours post-dose;
consumption of alcohol-based products from 24 hours prior to dosing until after the last sample collection;
food or beverages containing xanthine derivatives or xanthine-related compounds or energy drinks from 48 hours prior to dosing until after the last sample collection;
vitamins and natural food supplements from 7 days prior to dosing until after the last sample collection; and
grapefruit products from 7 days prior to dosing until after the last sample collection.

Subjects were asked to avoid consuming foods or beverages with a high concentration of vitamin B6 for 48 hours prior to dosing and until after the last sample collection of the study. Foods and beverages avoided were soya-based products, whole wheat and wheat bran products, banana, nuts, potato, carrot juice, prune juice, malted milk, fish, and chicken liver.

Non-surgically sterile males or males with partners of childbearing potential must have been willing to use condoms with a spermicide during study and for 14 days following the last study drug administration, and/or ensure that their partner(s) use effective contraception for the same time duration.

The number of cigarettes smoked was documented throughout the confinement period to ensure that subjects do not smoke more than 25 cigarettes per day while in-house.

Female subjects of childbearing potential and who have sexual intercourse with a non-sterile male partner, were required to use an acceptable method of contraception prior to study drug administration until 14 days following the last study drug administration. The accepted methods of contraception are listed above.

Subject Screening Procedures—Subjects were screened within 28 days preceding administration of the study medication for: demographic data, medical and medication histories, physical examination, body measurements (e.g. height, weight and body frame), ECG, vital signs, hematology, biochemistry, HIV, hepatitis B and C, urinalysis, urine drug screen, and urine pregnancy test.

Study Medication—Pyridoxal-5′-Phosphate Monohydrate enteric-coated tablet, total dose 250 mg (CanAm Bioresearch Inc., Canada).

Confinement, Visits and Dosing—Subjects were confined from at least 12 hours before dosing until after the 24.0-hour postdose blood draw. Subjects will return for a subsequent blood draw. Study medication was administered to each subject with 300 mL of water and a mouth check was performed to ensure consumption of the medication.

Concomitant Medication—No concomitant drug therapy was allowed during the study except one(s) used due to an adverse event. Any concomitant medication use, other than the use of hormonal contraceptives and the occasional use of acetaminophen, was evaluated on a case-by-case basis by the qualified investigator or a physician.

Blood Sample Collection and Processing—A total of 19 blood samples was drawn from each subject for quantitation of P5P, PAL (pyridoxal) and PA (4-pyridoxic acid). Blood samples were collected in EDTA blood tubes at 10, 1 and 0.25 hours prior to drug administration and 1.00, 2.00, 3.00, 3.50, 4.00, 4.50, 5.00, 5.50, 6.00, 7.00, 8.00, 10.0, 12.0, 14.0, 24.0, and 36.0 hours post-dose (10 mL for each sampling time). Unless otherwise specified or for subject safety, when blood draws and other procedures coincide, blood draws have precedence. When possible, blood samples were collected via a dead-volume intravenous catheter from pre-dose to 14 hours post-dose (or later); when not collected via catheter, blood samples were collected via direct venipuncture.

The total volume of blood including that collected for eligibility and safety purposes did not exceed 237 mL per subject for each cohort. Deviations of blood volume were reported only when the total volume of the whole study is exceeded.

Urine Sample Collection and Processing—For quantitation of P5P, PAL and PA, urine samples were collected at 6 times or time intervals: at pre-dose and 0.00-4.00, 4.00-8.00, 8.00-12.0, 12.0-24.0, and 24.0-36.0 hours post-dose. The pre-dose samples were obtained within 2 hours prior to dosing. Subjects were asked to void their bladder within 15 minutes before dosing, so they began the following interval with an empty bladder, and within 15 minutes from the end of the 12.0-24.0-hours interval. For time interval 24.0-36.0 hours post-dose, subjects were provided with urine containers and will be asked to collect all their urine at home and document the time of urine collection. Subjects received instructions for proper collection and storage of urine samples. They were asked to bring back all urine samples to the clinic at the scheduled return visit. Urine was also collected during the return visit (within 15 minutes from the end of the interval). Any urine voided by subjects at the intersection (within 10 minutes) of two intervals was included in the earlier sample. Any urine voided by subjects but not collected was documented.

Food and Fluid Intake—No food was allowed from at least 10 hours before dosing until at least 4 hours after dosing. Controlled meals were served at appropriate time thereafter. During confinement, all meals had controlled vitamin B6 content. Foods and beverages with a high concentration of vitamin B6 (see above) were avoided. Except for water given with study medication, no fluids were allowed from 2 hours before dosing until 2 hours post-dose. Water was provided ad libitum at all other times.

Subject Safety—Subjects were monitored throughout the study for adverse events to the study medication and/or procedures. Blood pressure, heart rate, respiratory rate and oral temperature was measured in sitting position (except for safety reasons) prior to and approximately 1, 2, 4, 6, and 12 hours after dosing (when vital signs measurements coincide with a blood draw, they were preferably performed before the blood collection whenever possible). Supine ECG was performed prior to and approximately 1, 2, 4, 6, and 12 hours after dosing (when ECG coincide with a blood draw, they were preferably performed as soon as possible after the blood collection whenever possible). Vital signs measurement were repeated at least once under the following conditions:
1) scheduled systolic blood pressure measurement lower than 90 mmHg, or higher than 140 mmHg;
2) scheduled diastolic blood pressure measurement lower than 50 mmHg, or higher than 90 mmHg;
3) scheduled heart rate lower than 50 bpm, or higher than 100 bpm; or
4) upon physician's request. The physician must be notified of all repeated vital sign measurements that are still outside the normal range values mentioned above to evaluate the significance of the results and decide further action if needed.

Hematology, biochemistry, urinalysis, urine drug screen, and serum pregnancy test were performed before drug administration.

Post-Study Procedures—Hematology, biochemistry, urinalysis, physical examination, vital signs, ECG, urine pregnancy test, and adverse event monitoring were performed on the last study day or up to 14 days after the last participation of the subject in the study.

Safety and Tolerance Parameters—Safety and tolerance were evaluated through the assessment of adverse events, vital signs, 12-lead ECG, clinical laboratory parameters and physical examination. Adverse events were tabulated.

Pharmacokinetic Parameters—The following pharmacokinetic parameters were calculated by standard non-compartmental methods for P5P, PAL and PA:
Plasma samples were used to calculate the following parameters:
   1) Cₘₐₓ: maximum observed concentration,
   2) Tₘₐₓ: time of observed Cₘₐₓ,
   3) Kₑₗ: elimination rate constant,
   4) T_{1/2 el}: elimination half-life,
   5) AUC₀₋ₜ: area under the concentration-time curve from time zero to the last non-zero concentration,
   6) AUC_{0-inf}: area under the concentration-time curve from time zero to infinity (extrapolated),
   7) AUC_{t/inf}: ratio of AUC0-t to AUC0-inf,
   8) Cl/F: total body clearance, calculated as Dose/AUC0-inf,
   9) V_{d}/F: apparent volume of distribution, calculated as Dose/(Kel x AUC0-inf), and
   10) MRT: mean residence time
Urine samples will be used to calculate the following parameter:
   1) Cumulative urinary excretion (Ae₀₋ₜ).

Statistical Analyses—Descriptive statistical analyses were performed.

Baseline correction: Since P5P has endogenous concentrations coming from vitamin B6 intake, it was analyzed both with and without baseline correction. Each subject was corrected for the mean results of the plasma samples taken pre-dose (−10, −1 and −0.25 hours prior to drug administration) for that same subject. If, after correction, any negative concentrations result, they were set equal to zero

Results—The average concentrations of plasma and urine P5P, PAL. And PA following a single 250 mg dose of enteric coated P5P are set out in Table 2.

TABLE 2
Concentration of Plasma and Urine P5P and Metabolites

| | | |
|---|---|---|
| Sample Analyzed | Cmax (ng/ml +/− SE) | Tmax (hr +/− SE) |

| | | | |
|---|---|---|---|
| Plasma P5P | 371.3 +/− 86.4 | 3.2 +/− 0.5 | |
| Plasma PAL | 1971.0 +/− 113.8 | 3.8 +/− 0.5 | hr |
| Plasma PA | 3372.3 +/− 314.4 | 3.8 +/− 0.5 | hr |
| Urine P5P | 325.0 +/− 32.3 | 4.0-36.0 | hr |
| Urine PAL | 5255.1 +/− 999.6 | 0.0-8.0 | hr |

The only adverse events reported during the study were one report of mild drowsiness at 8:55 in the morning and one report of mild pain at the coccyx area at 12:00 noon. The infrequency and mildness of the reported adverse events suggest that P5P is safe and tolerable at a 250 mg dose.

Two of six patients reported adverse events on the day of dosing (250 mg orally at 7:00 am). One subject reported mild drowsiness at 8:55 am and one subject reported mild pain at the coccyx area at 12:00 noon. Drowsiness was considered by the investigator to have possible relationship to drug treatment, whereas pain at the coccyx area was considered to have no relationship to the drug. The infrequency and mildness of the reported adverse events suggest that P-S-P is safe and tolerable at a 250 mg oral dose.

What is claimed:

1. A pyridoxal-5′-phosphate pharmaceutical formulation suitable for oral administration comprising a dissolution profile, when measured in a standard dissolution apparatus, according to the United States Pharmacopoeia dissolution test, at 37° C. in a 0.05M phosphate buffered solution having a pH of 6.8 at 75 rpm, as follows:
a) greater than about 30% at 15 minutes,
b) greater than about 85% at 30 minutes,
c) greater than about 90% at 45 minutes, or
d) greater than about 95% at 60 minutes.

2. A pyridoxal-5′-phosphate pharmaceutical formulation comprising a dissolution profile, when measured in a standard dissolution apparatus, according to the United States Pharmacopoeia dissolution test, at 37° C. in 0.1N HCl at 75 rpm, of up to 10% in 120 minutes.

3. A pyridoxal-5-phosphate pharmaceutical formulation wherein the in vivo oral intake of between 15 and 60 mg/kg produces a maximum plasma level (Cₘₐₓ) of pyridoxal-5′-phoshpate of between about 1 and about 8 mg/L.

4. The pharmaceutical formulation according to claim 1, wherein the tablet comprises: (a) a core, wherein said core comprises pyridoxal-5′-phosphate or pharmaceutically acceptable salt thereof, (b) a sub-coat surrounding the core; (c) an enteric coat surrounding the sub-coat; and, optionally, (d) a color coat surrounding the enteric coat

5. The pharmaceutical formulation according to claim 4, wherein the core further comprises a disintegrant or mixtures of disintegrants.

6. The pharmaceutical formulation according to claim 5, wherein the disintegrant or mixture of disintegrants comprise microcrystalline cellulose.

7. The pharmaceutical formulation according to claim 6, wherein the microcrystalline cellulose has a particle size of about 0.100 mm.

8. The pharmaceutical formulation according to claim 7, wherein the microcrystalline cellulose is Avicel PH 102.

9. The pharmaceutical formulation according to claim 4, wherein the core further comprises povidone.

10. The pharmaceutical formulation according to claim 9, wherein the povidone has a K value of between 27-33.

11. The pharmaceutical formulation according to claim 10, wherein the povidone is PVP K30.

12. The pharmaceutical formulation according to claim 4, wherein the sub-coat is Opadry®-IR-7000 White.

13. The pharmaceutical formulation according to claim 12, wherein the amount of Opadry®-IR-7000 White is about 3% w/w

14. The pharmaceutical formulation according to claim 4, wherein the enteric coat is Acryl-EZE White.

15. The pharmaceutical formulation according to claim 14, wherein the amount of Acryl-EZE White is about 10 to 12% w/w.

16. The pharmaceutical formulation according to claim 15, wherein the amount of Acryl-EZE White is about 10% w/w.

17. The pharmaceutical formulation according to claim 4, wherein the core further comprises a lubricant.

18. The pharmaceutical formulation according to claim 17, wherein the lubricant is magnesium stearate.

19. The pharmaceutical formulation according to claim 4, wherein the disintegrant or disintegrant mixture comprises croscarmellose sodium.

20. The pharmaceutical formulation according to claim 4, wherein the disintegrant or disintegrant mixture comprises microcrystalline cellulose and croscarmellose sodium.

21. The pharmaceutical formulation according to claim 4, wherein the core further comprises talc.

22. The pharmaceutical formulation according to claim 4, wherein the core further comprises colloidal silicon dioxide.

23. The pharmaceutical formulation according to claim 1, wherein the formulation comprises at least 50% w/w pyridoxal-5′-phosphate.

24. The pharmaceutical formulation according to claim 23, wherein the formulation comprises between about 60% and about 70% w/w pyridoxal-5′-phosphate.

25. The pharmaceutical formulation according to claim 24, wherein the formulation comprises about 66.3% w/w pyridoxal-5′-phosphate.

26. The pharmaceutical formulation according to claim 4, comprising: about 65% to about 75% w/w pyridoxal-5′-phosphate or a pharmaceutically acceptable salt thereof, about 20 to 30% w/w microcrystalline cellulose, about 2.0% to about 4.0% w/w croscarmellose sodium, about 3.0% to about 6.0% w/w povidone, about 1.0% to about 4.0% w/w talc, about 0.1% to about 1.0% w/w colloidal silicon dioxide, and about 0.5% to about 1.5% w/w magnesium stearate.

27. The pharmaceutical formulation according to claim 3 where the Cₘₐₓ of pyridoxal-5-phosphate is between about 0.1 and about 2 mg/L.

28. The pharmaceutical formulation according to claim 4, comprising: about 66.3% w/w pyridoxal-5′-phosphate or a pharmaceutically acceptable salt thereof, about 21.6% w/w microcrystalline cellulose, about 4.0% w/w croscarmellose sodium, about 4.7% w/w povidone, about 2.0% w/w talc, about 0.5% w/w colloidal silicon dioxide, and about 1.0% w/w magnesium stearate.

29. The pharmaceutical formulation according to claim 4, wherein the color coat is Opadry® Blue Fx.

30. The pharmaceutical formulation according to claim 29, wherein the Opadry® Blue Fx is an about 7.5 w/v dispersion of Opadry® Blue Fx.

31. The pharmaceutical formulation according to claim 29, wherein the Opadry® Blue Fx is an about 7.5 w/v dispersion of Opadry® Blue Fx.

32. A pre-blend for the manufacture of a pyridoxal-5′-phosphate oral dosage form comprising: at least about 50% w/w pyridoxal 5′-phosphate.

33. The pre-blend according to claim 32, wherein the pre-blend further comprises microcrystalline cellulose.

34. The pre-blend according to claim 33, wherein the pre-blend further comprises croscarmellose sodium.

35. The pre-blend according to claim 34 comprising: about 82.7% w/w pyridoxal 5′-phosphate, about 14.8% w/w microcrystalline cellulose, and about 2.5 % w/w croscarmellose sodium.

36. The pre-blend according to claim 33, wherein the microcrystalline cellulose has a particle size of about 0.100 mm.

37. The pre-blend according to claim 33, wherein the microcrystalline cellulose is Avicel PH 102.

38. The pre-blend according to claim 32, wherein the pre-blend further comprises a povidone having a K value of between 27-33.

39. The pre-blend according to claim 38, wherein the povidone is PVP K-30.

40. A method of preparing an oral dosage form of pyridoxal-5′-phosphate comprising the steps of:
a) dissolving a granulation binder in purified water to provide a granulating solution;
b) mixing at least 50% w/w/pyridoxal-5′-phosphate or pharmaceutically acceptable salt with a disintegrant or a mixture of disintegrants to provide a pre-blend;
c) mixing the pre-blend with the granulating solution to provide a granulating preparation;
d) substantially drying the granulating preparation;
e) mixing excipients with the granulating preparation to provide a semi-final blend preparation;
f) mixing the semi-final blend preparation with a lubricant to provide a final blend preparation;
g) compressing the final blend preparation into a core;
h) applying a sub-coat to the core to provide a sub-coated core; and
i) applying an enteric coat to the sub-coated core.

41. The method according to claim 40, wherein the disintegrant or disintegrant mixture comprises microcrystalline cellulose.

42. The method according to claim 41, wherein the microcrystalline cellulose has a particle size of about 0.100 mm.

43. The method according to claim 41, wherein the microcrystalline cellulose is Avicel PH 102.

44. The method according to claim 40, wherein the disintegrant or disintegrant mixture comprises croscarmellose sodium.

45. The method according to claim 44, wherein the disintegrant or disintegrant mixture comprises microcrystalline cellulose and croscarmellose sodium.

46. The method according to claim 40, wherein the pre-blend comprises about 8.0% to about 20% w/w microcrystalline cellulose and about 1.0% to about 4.0% w/w croscarmellose sodium.

47. The method according to claim 40, wherein the granulation binder comprises povidone with a K value of between 27-33.

48. The method according to claim 40, wherein the granulation binder comprises about 4.7% w/w povidone.

49. The method according to claim 48, wherein the povidone is PVP K-30.

50. The method according to claim 40, wherein the sealing coat is an about 15% w/v dispersion of Opadry®l-IR-7000 White.

51. The method according to claim 40, wherein the enteric coat is an about 20% w/v dispersion of Acryl-EZE White.

52. The method according to claim 40, wherein the pyridoxal 5′-phosphate, the microcrystalline cellulose, and the croscarmellose are mixed with a high shear mixer to provide the pre-blend.

53. The method according to claim 52, wherein the pre-blend and the granulating solution are mixed by spraying the granulating solution onto the pre-blend while the pre-blend is being mixed in the high shear mixer.

54. The method according to claim 40, further comprising the step of passing the granulating preparation through a conical mill with a 0.5″ screen following step (c) and prior to step (d).

55. The method according to claim 40, wherein the granulating preparation is dried using a fluid bed dryer at 60° C.

56. The method according to claim 40, further comprising the step of passing the granulating preparation through a 20 mesh screen and then mixing the granulating preparation in a diffusive blender, following step (d) and prior to step (e).

57. The method according to claim 40, further comprising the steps of mixing the pre-blend preparation is using a small diffusive blender and passing the mixed pre-blend preparation through a 20 mesh screen.

58. The method according to claim 40, wherein the granulating preparation and pre-blends are mixed using a diffusive blender to make a semi-final blend preparation.

59. The method according to claim 40, further comprising the step of passing the lubricant through a 30 mesh screen prior to mixing the lubricant with the semi-final blend preparation.

60. The method according to claim 40, wherein the semi-final blend preparation and a lubricant are mixed using a diffusive blender.

61. The method according to any one of claims 40 to 60, wherein the lubricant is magnesium stearate.

62. The method according to claim 40, wherein the excipients of step (e) comprises colloidal silicon dioxide.

63. The method according to claim 40, wherein the excipients of step (e) comprises about 0.5% w/w colloidal silicon dioxide.

64. The method according to claim 40, wherein the excipients of step (e) comprises talc.

65. The method according to claim 40, wherein the excipients of step (e) comprises about 2% w/w talc.

66. The method according to claim 40, wherein the excipients of step (e) comprises a disintegrant or a mixture of disintegrants.

67. The method according to claim 66, wherein the disintegrant or a mixture of disintegrants comprises microcrystalline cellulose.

68. The method according to claim 66, wherein the disintegrant or a mixture of disintegrants comprises croscarmellose sodium.

69. The method according to claim 66, wherein the disintegrant or a mixture of disintegrants comprises microcrystalline cellulose and croscarmellose sodium

70. The method according to claim 40, wherein the excipients of step (e) comprises about 8.0% to about 20% w/w microcrystalline cellulose and about 1.0% to about 4.0% w/w croscarmellose sodium.

71. The method according to claim 40, wherein the excipients of step (e) comprises about 8.0% to about 12.0% w/w microcrystalline cellulose, about 1.0% to about 4.0% w/w croscarmellose sodium, about 1.0% to about 4.0% w/w talc, and about 0.1 % to about 1.0% w/w colloidal silicon dioxide.

72. The method according to claim 40, wherein the pyridoxal-5′-phosphate or a pharmaceutically acceptable salt thereof is between about 60% and about 70% w/w.

73. The method according to claim 40, wherein the pyridoxal-5′-phosphate or a pharmaceutically acceptable salt thereof is about 66.3% w/w.

74. The method according to claim 40, wherein the steps comprise:
a) dissolving about 4.7% w/w povidone in purified water to provide a granulating solution;
b) mixing about 66.3% w/w pyridoxal-5′-phosphate or pharmaceutically acceptable salt with about 11.9% w/w microcrystalline cellulose and about 2.0% w/w croscarmellose sodium to provide a pre-blend;
c) mixing the pre-blend with the granulating solution to provide a granulating preparation;
d) substantially drying the granulating preparation;
e) mixing about 9.7% w/w microcrystalline cellulose, about 2.0% w/w croscarmellose sodium, about 2.0% w/w talc, and about 0.5% w/w colloidal silicon dioxide, to provide a pre-blend preparation;
f) mixing the granulating preparation and the pre-blend preparation to provide a semi-final blend preparation;
g) mixing the semi-final blend preparation with about 1.0% w/w magnesium stearate to provide a tableting preparation;
h) compressing the tableting preparation into a core;
i) applying a sub-coat to the core to provide a sub-coated core; and
j) applying an enteric coat to the sub-coated core.

75. A method of reducing the incidence of nausea and vomiting associated with the oral administration of pyridoxal 5′-phosphate or a pharmaceutically acceptable salt thereof, said method comprising the step of administering an effective amount of the pharmaceutical composition according to claim 1.

76. Use of a pharmaceutical composition according to claim 1 for reduction of the incidence of nausea and vomiting associated with the oral administration of pyridoxal 5′-phosphate or a pharmaceutically acceptable salt thereof.

77. A method of increasing patient compliance in a patient in need of treatment with pyridoxal-5-phosphate, comprising administering an effective amount of the pharmaceutical composition according to claim 1.

78. Use of a pharmaceutical composition according to claim 1 for increased patient compliance in a patient in need of treatment with pyridoxal-5-phosphate.
